(19) United States
(12) Patent Application Publication
(10) Pub. No.: US 2023/0384309 A1
(43) Pub. Date: Nov. 30, 2023

(54) SYSTEM AND METHOD FOR VIRUS DETECTION USING NANOPARTICLES AND A NEURAL NETWORK ENABLED MOBILE DEVICE

(71) Applicant: The Brigham and Women's Hospital, Inc., Boston, MA (US)

(72) Inventors: Mohamed Shehata DRAZ, Somerville, MA (US); Hadi SHAFIEE, Boston, MA (US)

(21) Appl. No.: 18/026,442

(22) PCT Filed: Sep. 15, 2020
(86) PCT No.: PCT/US2021/050453

§ 371 Date: Mar. 15, 2023

Related U.S. Application Data
(60) Provisional application No. 63/167,088, filed on Mar. 28, 2021.
(60) Provisional application No. 63/078,691, filed on Sep. 15, 2020.

(51) Int. Cl. G01N 33/569; C12Q 1/28; B01L 3/00; G06T 7/00; G16H 10/40; G16H 40/67; G16H 50/20

(52) U.S. Cl. CPC G01N 33/56983; C12Q 1/28; B01L 3/502761; G06T 7/0014; G16H 10/40; G16H 40/67; G16H 50/20; B01L 2200/0652; B01L 2200/16; G01N 2333/908; G01N 2469/10; B01L 2300/027; G06T 2207/20084; G06T 2207/30004; G06T 2207/20076

(57) ABSTRACT

A system for virus detection in a sample from a subject includes a microchip comprising at least one channel containing the sample from the subject and a mobile device. The sample is processed with nanoparticles and a catalyzer that are configured to generate gas bubbles in the presence of a target virus on a surface of the microchip. The mobile device includes a camera configured to acquire an image of the microchip containing the sample from the subject, a neural network configured to receive the acquired image and to generate a probability regarding the presence of the target virus in the sample from the subject based on the acquired image, and a display coupled to the neural network and configured to display the probability regarding presence of the target virus in the sample from the subject.

### CROSS-REFERENCE TO RELATED APPLICATIONS

This application is based on, claims priority to, and incorporates herein by reference in its entirety U.S. Ser. No. 63/078,691 filed Sep. 15, 2020, and entitled “Gas Bubble Sensing on Chip for Point of Care Diagnostics” and U.S. Ser. No. 63/167,088 filed Mar. 28, 2021, and entitled “Gas Bubble Sensing on Chip for Point-of-Care Diagnostics.”

### STATEMENT REGARDING FEDERALLY SPONSORED RESEARCH

This technology was made with government support under grants R01AI118502, R01AI138800, R61AI140489, and 5P30AI060354-14 awarded by the National Institutes of Health. The government has certain rights in the technology

### FIELD

The present disclosure relates generally to mobile health and point-of-care diagnostics and, more particularly, to systems and methods for virus detection using nanoparticles and neural network enabled mobile devices (e.g., smartphones, tablets, etc.).

### BACKGROUND

Emerging and reemerging infections present an ever-increasing challenge to global health. Rapid and sensitive point-of-care (POC) diagnostics with the ability to be seamlessly integrated with appropriate and effective surveillance mechanisms can shift the paradigm in outbreak control and the prevention of new epidemics. For example, nanoparticle-enabled digital health systems can help large-scale and rapid screening of infectious diseases.

Mobile health (mHealth) diagnostics are changing the face of modern medicine and healthcare. The growing advances in consumer electronics and portable communication systems, particularly mobile phones (e.g., smartphones), have led to significant growth of mobile phone subscribers worldwide particularly in developing countries and have led to faster and cheaper approaches of data acquisition and for developing point-of-care (POC) diagnostics. The global unique mobile subscribers in 2019 was approximately 5.18 billion and is estimated to reach more than 5.7 billion by 2025, and more that 10% of this number will be in regions of the world where most of infection outbreaks occur. Such global access to mobile phones, combined with its powerful computing ability and built-in sensors present a promising potential to develop digital diagnostics that may help in large scale and efficient management of infectious diseases.

Smartphone systems can also benefit from the most recent unprecedented advancements in nanotechnology to develop novel diagnostic approaches. Catalysis can be considered as one of the popular applications of nanoparticles because of their large surface-to-volume ratio and high surface energy. Numerous diagnostic platforms for cancer and infectious diseases have been developed by substituting enzymes, such as catalase, oxidase, and peroxidase with nanoparticle structures. Previous work in mobile health technologies for target virus/protein detection, however, lack the generalization of the technology and adaptability for different smartphone models due to the dependency on smartphone specific hardware optical attachments.

It would be desirable to provide a system and method for point-of-care diagnostics that allows for simple, rapid and sensitive virus detection.

### SUMMARY

In accordance with an embodiment, a system for virus detection in a sample from a subject includes a microchip comprising at least one channel containing the sample from the subject and a mobile device. The sample is processed with nanoparticles and a catalyzer that are configured to generate gas bubbles in the presence of a target virus on a surface of the microchip. The mobile device includes a camera configured to acquire an image of the microchip containing the sample from the subject, a neural network configured to receive the acquired image and to generate a probability regarding the presence of the target virus in the sample from the subject based on the acquired image, and a display coupled to the neural network and configured to display the probability regarding presence of the target virus in the sample from the subject.

In accordance with another embodiment, a method for virus detection in a sample from a subject includes loading the sample from the subject into a microchip comprising at least one channel, processing the sample from the subject using at least nanoparticles and a catalyzer that are configured to generate gas bubbles in the presence of a target virus, acquiring an image of the microchip containing the sample from the subject using a mobile device, providing the acquired image to a neural network, generating, using the neural network, a probability regarding the presence of the target virus in the sample from the subject based on the acquired image and displaying the probability regarding the presence of the target virus in the sample from the subject on a display.

### BRIEF DESCRIPTION OF THE DRAWINGS

The present disclosure will hereafter be described with reference to the accompanying drawings, wherein like reference numerals denote like elements.

FIG. 1 is a block diagram of a system for virus detection using nanoparticles and a neural network enabled mobile device in accordance with an embodiment;

FIG. 2 illustrates a method for virus detection using nanoparticles and a neural network enabled mobile device in accordance with an embodiment;

FIGS. 3A and 3B illustrate an example application of the method for virus detection using nanoparticles and neural network enabled mobile devices of FIG. 2 in accordance with an embodiment;

FIG. 4 illustrates an example convolution neural network architecture for classifying a sample of a subject for virus detection in accordance with an embodiment;

FIG. 5 illustrates an example process for preparing a nanooprobe solution in accordance with an embodiment;

FIG. 6 is a diagram illustrating an example microchip surface modification in accordance with an embodiment;

FIG. 7 illustrates an example structure of a target virus labeled with nanoparticles in accordance with an embodiment;

FIG. 8 illustrates an example microchip and sample before bubble formation and after bubble formation in accordance with an embodiment;

FIG. 9 shows example images of the formation of bubbles over time in accordance with an embodiment;

FIG. 10A is a perspective view of a sample processing cartridge in accordance with an embodiment;

FIG. 10B is an exploded view of the sample processing cartridge of FIG. 10A in accordance with an embodiment;

FIG. 11 is a diagram of a sample processing cartridge including reagents and materials for processing a sample of a subject in accordance with an embodiment; and

FIG. 12 illustrates a method for virus detection using nanoparticles and a neural network enabled mobile device including processing a microchip with a sample of a subject using the sample processing cartridge of FIG. 11 in accordance with an embodiment.

### DETAILED DESCRIPTION

The present disclosure describes systems and methods for rapidly detecting a biological or other residue in a sample (e.g., blood, serum, plasma) from a subject (e.g., a patient). In some embodiments, the described systems and methods may be used to provide rapid and sensitive point-of-care diagnostics particularly for infectious diseases. In particular, the present disclosure describes systems and methods for rapid and sensitive virus detection using nanoparticles and a deep neural network-enabled mobile device (e.g., a smartphone, tablet, etc.). In some embodiments, a sample from a subject may be loaded on a microchip that is configured to capture a target virus, for example, the surface of the microchip may be modified with a probe material such as, for example, monoclonal antibodies against a target virus protein. The sample may then be processed using nanoparticles and a catalyzer that can be loaded onto the microchip. In some embodiments, the nanoparticles are designed to induce gas bubbles formation in the presence of the catalyzer and the target virus. The bubbles formed may be correlated with the concentration level of the target virus in the sample In some embodiments, the formed bubbles may make distinct visual patterns based on the presence of the target virus (e.g., the concentration of target virus (or viral load)) in the sample. An image of the microchip (and any formed bubbles) may be acquired using a mobile device and classified using a neural network on the mobile device. In some embodiments, the neural network on the mobile device may be configured to qualitatively detect the presence of the target virus in the sample, for example, the neural network may generate a probability value of the sample as being positive or negative for the target virus based on the acquired image of the microchip and sample. The target virus may be, for example, a Zika virus (ZIKV), hepatitis B virus (HBV), hepatitis C virus (HCV), dengue virus (DENY-1 and -2), human cytomegalovirus (HCMV), herpes simplex virus (HSV), etc. In some embodiments, the mobile device is a mobile phone such as, for example, a smartphone and the neural network is a convolution neural network (CNN). In some embodiments, the nanoparticles used to process the sample are metal nanoparticles and the intrinsic catalytic properties of the metal nanoparticles are adopted for gas bubble formation to detect viruses on-chip using a convolutional neural network (CNN)-enabled smartphone system. The metal nanoparticles may be, for example, platinum (Pt) nanoparticles, gold (Au) nanoparticles, copper (Cu) nanoparticles, iron (Fe) nanoparticles, palladium (Pd) nanoparticles, zinc (Zn) nanoparticles, cadmium (Cd) nanoparticles, silver (Ag) nanoparticles, and other metal nanoparticles. The catalyzer can be, for example, a solution including hydrogen peroxide (H₂O₂). The disclosed systems and methods provide visual signal amplification through on-chip bubble formation combined with a neural network on a mobile device which advantageously allows simple and rapid virus detection using a mobile device camera without the need for any external mobile device optical attachment for image magnification and readout or any target amplification.

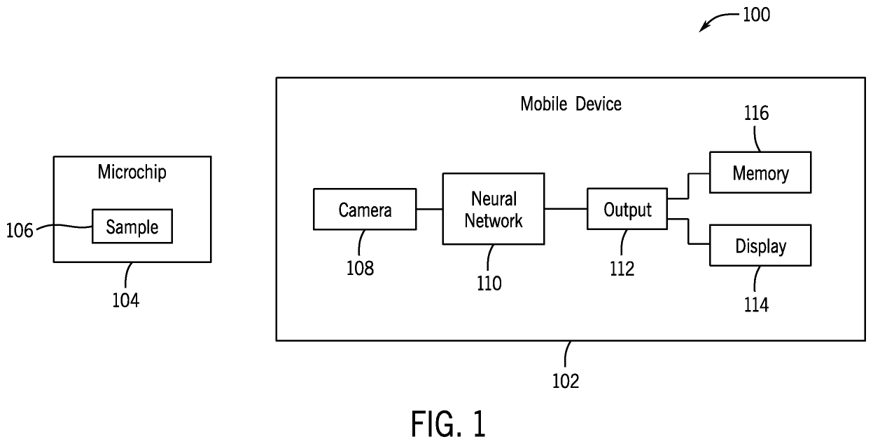

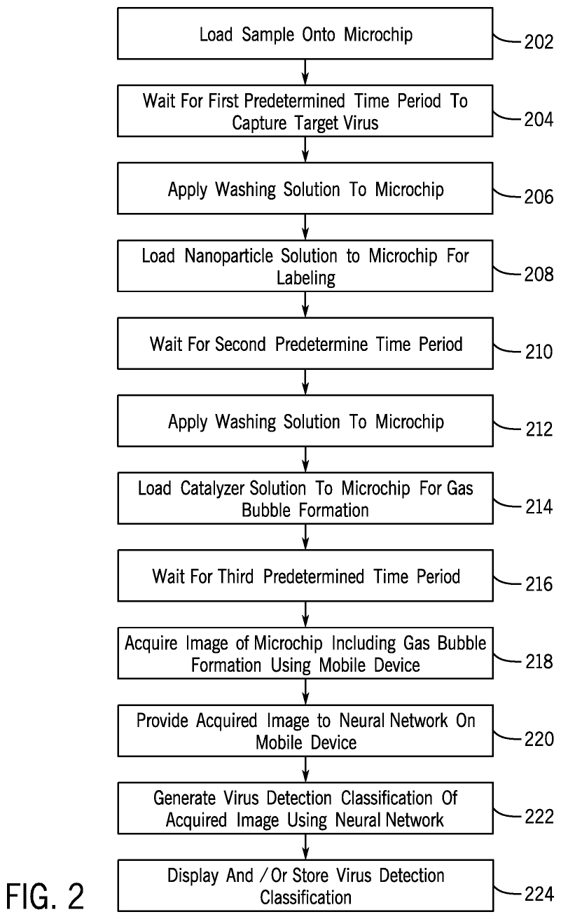

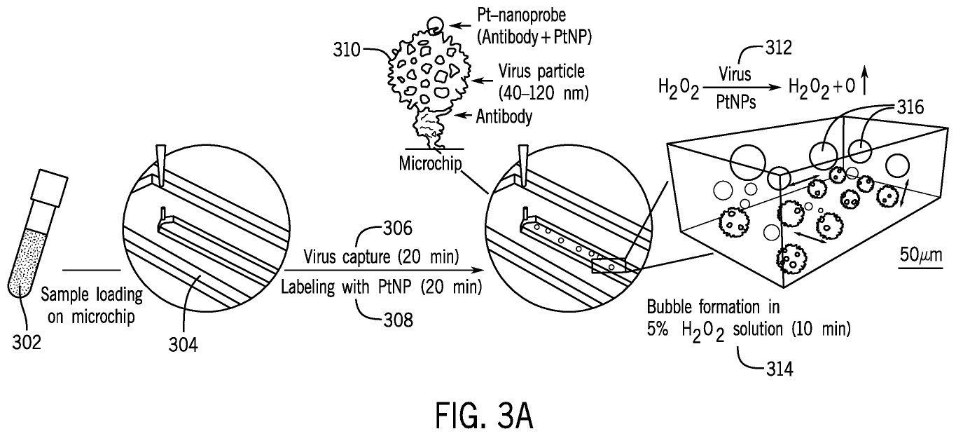

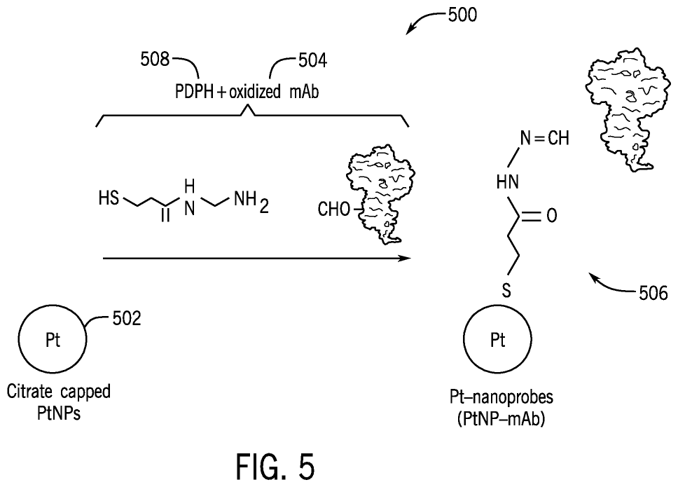

FIG. 1 is a block diagram of a system for virus detection using nanoparticles and neural network enabled mobile devices in accordance with an embodiment. The system 100 includes mobile device 102 and a microchip 104. The microchip 104 is configured to receive a sample 106 (e.g., blood, plasma/serum, serum) from a subject to be tested to determine if the sample is infected with a target virus. The target virus may be, for example, a Zika virus (ZIKV), hepatitis B virus (HBV), hepatitis C virus (HCV), dengue virus (DENY-1 and -2), human cytomegalovirus (HCMV), herpes simplex virus (HSV), etc. In some embodiments, the microchip 104 is a single channel microchip and the sample 106 is loaded into the microchip 104 (e.g., using a pipette). In some embodiments, the single channel microchip 304 may be fabricated from glass slides and layers of poly(methyl methacrylate) (PMMA), and double-sided adhesive film (DSA). In some embodiments, the surface of the microchip may be modified using a probe material to capture any virus particles in the sample 106 as discussed further below with respect to FIG. 6. In some embodiments, the probe material may be, for example, monoclonal antibody (mAb) against a target virus protein, DNA/RNA probes, aptamer, etc. In addition, the sample 106 can be processed on the microchip 104 using, for example, nanoparticles and a catalyzer, to allow for the formation of bubbles on the surface of the microchip 104 in the presence of a target virus in the sample as described further below with respect to FIGS. 2, 3A and 3B. The mobile device 102 (e.g., a smartphone, a tablet, etc.) may be configured to acquire an image of the microchip 104 and the sample 106 and to analyze the image using a neural network to determine whether the sample 106 is infected or not infected with the target virus based on the bubble formation on the microchip 104 as discussed further below with respect to FIGS. 2, 3A and 3B.

The mobile device 102 includes a camera 108, a neural network 110, an output 112 of the neural network 110, a display 114 and a memory 116. The camera 108 may be configured to allow a user of the mobile device 102 to acquire an image of the microchip 104 and sample 106. Advantageously, the image pf the microchip 104 and sample 106 may be directly acquired by the camera 18 of the mobile device 102 without an optical attachment for the mobile device 102. The acquired image of the microchip 104 and sample 106 may be input into the neural network 110 which is configured and trained to generate an output 112 indicating whether the sample 106 is infected (i.e., positive) or not infected (i.e., negative) based on the acquired image of the microchip 104 and sample 104. The neural network may be trained using known methods. In some embodiments, the neural network 110 is a convolutional neural network (CNN) such as, for example, an Inception v3 architecture, that may be pre-trained using the ImageNet image database. The pre-trained CNN may then be fine-tuned with a training data set that includes pre-labeled images of bubble formations or patterns on microchips using, for example, various target viruses, target virus concentrations, and different dilutions of nanoparticles (e.g., platinum nanoparticles (PtNPs)). In some embodiments, the neural network output 112 is a probability value of the sample 106 being positive or negative for the target virus. The output 112 may be displayed on a display 114 of the mobile device 102. The output 112 may also be stored in the memory 116 of the mobile device 102.

In some embodiments, the mobile device 102 may be, for example, a mobile phone, a smartphone, a tablet, or the like, or other standalone optical systems for imaging. As such, the mobile device 102 may include any suitable hardware and components designed or capable of carrying out a variety of processing and control tasks, including steps for acquiring an image of the microchip using camera 108, implementing the neural network 110, providing the output 112 to the display or storing the output 112 in memory 116. For example, the mobile device 102 may include a programmable processor or combination of programmable processors, such as central processing units (CPUs), graphics processing units (GPUs), and the like. In some implementations, the mobile device 102 may be configured to execute instructions stored in a non-transitory computer readable-media. In this regard, the mobile device 102 may be any device or system designed to integrate a variety of software, hardware, capabilities and functionalities. Alternatively, and by way of particular configurations and programming, the mobile device 102 may include a special-purpose system or device. For instance, such special-purpose system or device may include one or more dedicated processing units or modules that may be configured (e.g., hardwired, or pre-programmed) to carry out steps, in accordance with aspects of the present disclosure.

While the following description of FIGS. 2-12 may be discussed in terms of using, as an example, platinum (Pt) nanoparticles, it should be understood that the systems and methods described herein may utilize other types of metal nanoparticles including, but not limited to, gold (Au), copper (Cu), iron (Fe), palladium (Pd), zinc (Zn), cadmium (Cd), and silver (Ag).

FIG. 2 illustrates a method for virus detection using nanoparticles and neural network enabled mobile devices in accordance with an embodiment. The process illustrated in FIG. 2 is described below as being carried out by the system for virus detection 100 as illustrated in FIG. 1. At block 202, a sample 106 from a subject may be loaded onto a microchip 104. In some embodiments, the microchip 104 may be a single channel microchip and the sample 106 can be loaded into the microchip 104 (e.g., manually using a pipette). As mentioned above, the surface of the microchip can be modified with a probe material such as, for example, monoclonal antibody against a target virus protein (e.g., anti-Zika virus envelope antibody for ZIKV, anti-Hepatitis B Virus Surface Antigen antibody for HBV, and anti-Hepatitis C Virus Core antibody for HCV) to allow efficient capture of particles of the target virus on the surface of the microchip 104. At block 204, the microchip 104 and sample 106 can be incubated for a first predetermined period of time to allow for capture of particles of the target virus if the target virus is present in the sample 106. In one example, the first predetermined time period (or incubation period) can be twenty minutes. An example microchip surface modification is discussed further below with respect to FIG. 6. At block 206, a washing solution (e.g., phosphate buffer (PB, pH 7.4)) is applied to the microchip 104 and sample 106.

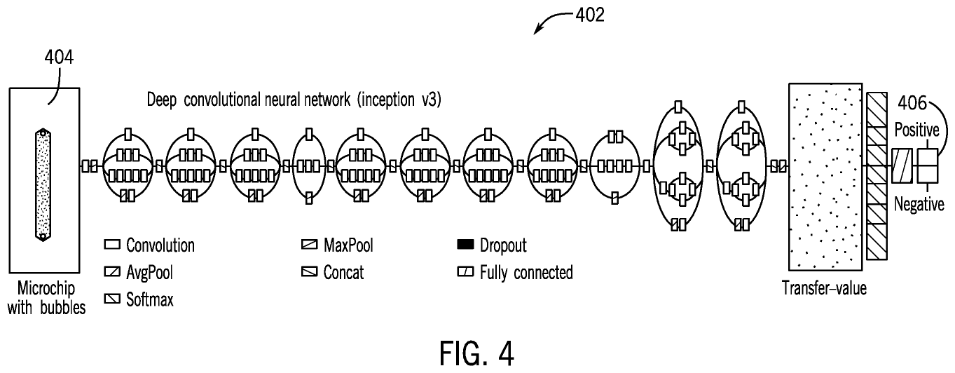

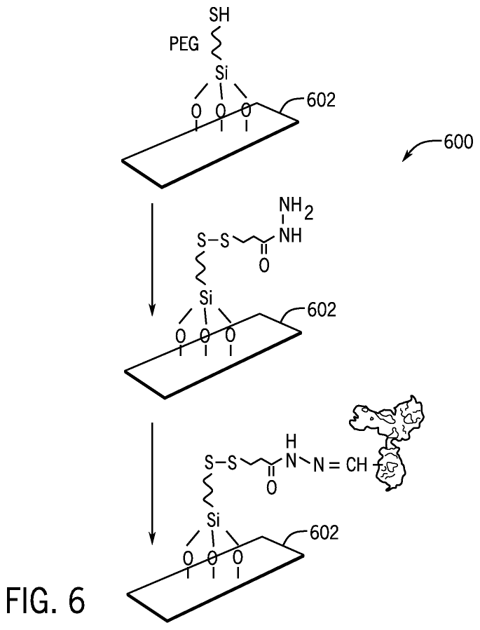

At block 208, a nanoparticle solution can be loaded onto the microchip 104 for labeling of any captured virus particles. In some embodiments, the nanoparticles may be metal nanoparticles including, but not limited to, platinum (Pt), gold (Au), copper (Cu), iron (Fe), palladium (Pd), zinc (Zn), cadmium (Cd), and silver (Ag). In some embodiments, the nanoparticle solution is a nanoprobe solution that includes nanoparticles modified with a probe material such as, for example, antibodies (e.g., monoclonal antibody (mAb) against a target virus), DNA/RNA probes, aptamer, etc. For example, the nanoparticles may be platinum nanoparticles (PtNPs) and the nanoparticle solution is a Pt-nanoprobe solution that includes PtNPs modified with monoclonal antibody (mAb) against a target virus. An example process for preparing a Pt-nanooprobe solution is described further below with respect to FIG. 5. At block 210, the microchip 104, sample 106, and nanoparticles (e.g., provided using a nanoprobe solution) can be incubated for a second predetermined period of time to allow any captured particles of the target virus in the sample 106 to be labeled by the nanoparticles. The labeling of captured virus particles with the nanoparticles can form virus immunocomplexes on the surface of the microchip 104, for example, target virus particles labeled with PtNPs can form Pt-virus immunocomplexes. An example structure of a target virus labeled with nanoparticles is described further below with respect to FIG. 7. In one example, the second predetermined time period (or incubation period) can be twenty minutes. At block 212, a washing solution (e.g., phosphate buffer (PB, pH 7.4)) is applied to the microchip 104 and sample 106 to, for example, wash any excess Pt-nanoprobes.

At block 214, a catalyzer solution can be loaded onto the microchip 104 to cause the formation of gas bubbles (i.e., bubble signal) if labeled target virus particles are present on the microchip 104. In some embodiments, the catalyzer solution includes hydrogen peroxide (H₂O₂). In the presence of captured Pt-virus immunocomplexes, bubbles can be formed due to the catalytic activity of PtNPs in contact with H₂O₂. High catalytic activity of PtNPs in high concentrations of H₂O₂, however, can lead to rapid merging of the generated bubbles on the surface of the microchip 104 and form irregular bubble shapes which can make accurate signal detection difficult. In some embodiments, to help avoid rapid bubble merging and to help control the stability of the visual patterns on-chip after virus capture and signal amplification, glycerol can be included in the catalyzer solution to increase the density of the catalyzer solution. In some embodiments, the catalyzer solution includes 5% H₂O₂ and 20% glycerol. At block 216, the microchip 104, sample 106, nanoparticles and catalyzer solution can be incubated for a third predetermined period of time to allow for bubble formation on the surface of the microchip 104 if there are labeled virus particles on the surface of the microchip 104. In some embodiments, the third predetermined time period is ten minutes.

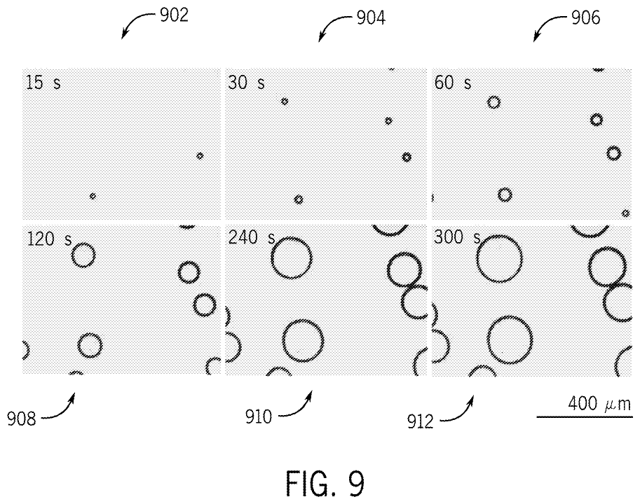

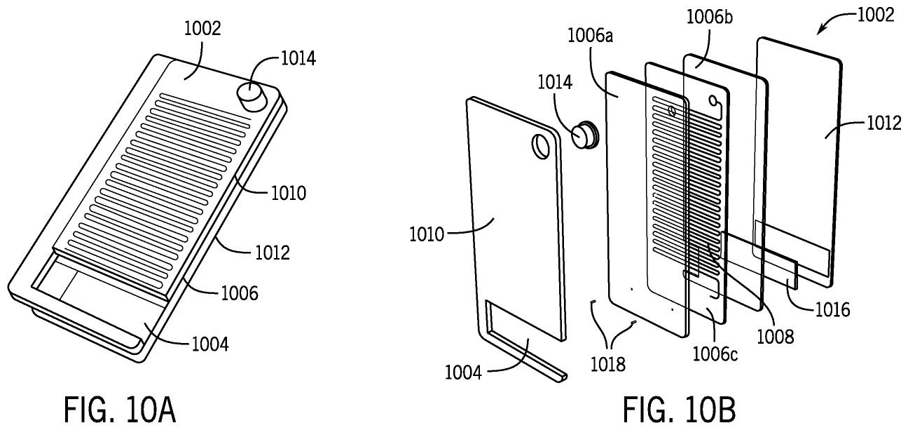

In come embodiments, the portions of the bubble assay protocol of blocks 206 to block 216 may be performed manually, for example, by loading and removing the various reagents and materials onto the microchip 104 using a pipette. In some embodiments, the portions of the bubble assay protocol of blocks 206 to block 216 may be performed using a sample processing cartridge that can be preloaded with the reagents and materials needed for sample processing (e.g., including washing solution, nanoprobe solution, catalyzer solution) as described further below with respect to FIGS. 10A-12.

At block 218 after the third predetermined time period, an image of the microchip 104 and sample 106, including any gas bubble formations may be acquired using a mobile device 102, for example, using a camera 108 of the mobile device 102. At block 220, the acquired image from block 218 may be provided to a neural network 110 on the mobile device 102 In some embodiments, the neural network can be trained to generate an output 112, for example, a virus detection classification, indicating whether the sample 106 is infected (i.e., positive) or not infected (i.e., negative) based on the acquired image of the microchip 104 and sample 104. The neural network may be trained using known methods. In some embodiments, the neural network 110 is a convolutional neural network (CNN) such as, for example, an Inception v3 architecture, that may be pre-trained using the ImageNet image database. The pre-trained CNN may then be fine-tuned with a training data set that includes pre-labeled images of bubble formations or patterns on microchips using, for example, various target viruses, target virus concentrations, and different dilutions of nanoparticles (e.g., PtNPs). At block 222, the neural network generates the virus detection classification of the acquired image. For example, in some embodiments, the neural network generates a probability value of the sample 106 being positive or negative for the target virus. At block 224, the output 112 of the neural network 110 (e.g., the generated probability value(s) and the acquired image of the microchip 104 and sample 106) may be displayed on a display 114 of the mobile device 102 and/or stored in the memory 116 of the mobile device 102.

FIGS. 3A and 3B illustrate an example application of the method for virus detection using nanoparticles and neural network enabled mobile devices of FIG. 2 in accordance with an embodiment. In FIG. 3A, a sample 302 from a subject is loaded onto a single channel microchip 304. In some embodiments, the single channel microchip 304 may be fabricated from glass slides and layers of poly(methyl methacrylate) (PMMA), and double-sided adhesive film (DSA). In this example embodiment, the target virus is Zika virus (ZIKV) and the microchip is modified with a monoclonal antibody (mAb) against the virus envelope protein. The sample may be loaded onto the microchip 304 using, for example, a pipette. In this example embodiment, the microchip 304 and sample on the microchip are incubated for 20 minutes to allow the capture 306 of any ZIKV particles in the sample. The virus capture 306 may be followed by a washing step by loading, for example, 10 mM phosphate buffer (pH 7.4) onto the microchip 304. A PtNP solution (e.g., a Pt-nanoprobe solution) may then loaded onto the microchip 304 for labeling any captured ZIKV particles. In this example embodiment, the microchip 304, sample and PtNP solution on the microchip are incubated for 20 minutes to allow the labeling 308 of any captured ZIKV particles on the surface of the microchip 304. The labeling with PtNP 308 may be followed by a washing step by loading, for example, 10 mM phosphate buffer (pH 7.4) onto the microchip 304. The labeling of captured ZIKV particles with PtNP forms Pt-virus immunocomplexes 310 on the surface of the microchip 304. Catalyzer solution 312 (e.g., containing hydrogen peroxide (H₂O₂) and glycerol) may then be added to the microchip 304 and, in this example embodiment, incubated for ten minutes to allow for the formation of gas bubbles 316. As mentioned above, in the presence of captured Pt-virus immunocomplexes, bubbles 316 can be formed due to the catalytic activity of PtNPs in contact with H₂O₂. In FIG. 3B, in this example embodiment, a smartphone 320 is used to acquire an image of the microchip 322, for example, the image may be acquired using a camera of the smartphone 320. The smartphone 320 can include a neural network (e.g., a CNN) that is configured to analyze the acquired image of the microchip 320 and generate an output, for example, a virus detection classification, indicating whether the sample 302 is infected (i.e., positive) or not infected (i.e., negative) For example, in some embodiments, the neural network generates a probability value of the sample 302 being positive or negative for the target virus. The smartphone 320 may also be configured to display the output of the neural network, for example, as shown in screenshots 330 and 332. In screenshot 330, an acquired image 338 of a microchip and probability values 334 determined by the neural network on the smartphone 320 are displayed. The probability values 334 include a probability the sample is positive (e.g., a viral load above or equal to a predetermined virus concentration threshold) for ZIKV and a probability the sample is negative (e.g., a viral load below the predetermined viral concentration threshold) for ZIKV. In some embodiments, the a threshold for probability values may be used to determine the classification for the sample. For example, if the probability value threshold is 0.5, the sample in image 330 may be classified as positive because the positive probability value of the sample is 0.7832114 (above the 0.5 threshold) and the negative probability value of the sample is 0.21678858 (below the 0.5 threshold). In screenshot 332, an acquired image 340 of a microchip and a probability value 336 determined by the neural network on the smartphone 320 are displayed. The probability value 334 includes a probability the sample is negative for ZIKV In this example, if the probability value threshold is 0.5, the sample in image 330 may be classified as negative because the negative probability value of the sample is 0.96054834 (above the 0.5 threshold).

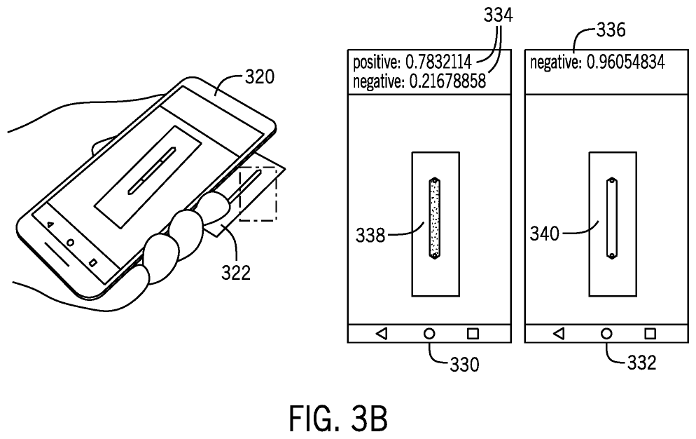

As discussed above, the neural network of the mobile device (e.g., a smartphone, tablet, etc.) may be a deep learning CNN. FIG. 4 illustrates an example convolution neural network architecture for classifying a sample of a subject for virus detection in accordance with an embodiment. In FIG. 4, a CNN model 402 is shown that is trained to analyze bubbles formed on an image of a single channel microchip with a sample from a subject to qualitatively identify samples as, for example, positive or negative for the target virus as discussed above. In some embodiments, the CNN model 402 may be configured to perform supervised learning to automatically recognize differences between two classes of positive (infected) and negative (non-infected) samples. The example CNN model 402 illustrated in FIG. 4 uses the Inception v3 architecture. In some embodiments, the CNN 402 may be pre-trained using the ImageNet image database, for example, a dataset of 1,000 object classes containing 1.28 million images of the 2014 ImageNet Challenge. In an embodiment, transfer learning may then be performed by removing the final classification layer from the CNN 402 and re-training (or fine tuning) the CNN 402 with a training dataset of images of microchips containing bubbles analogous to virus samples. For example, the raining dataset for fine tuning the CNN 402 may include pre-labeled images of single-channel microchips with bubbles (e.g., formed from various target viruses, target virus concentrations, and different dilutions of nanoparticles) and organized in the two different classes (positive and negative) for training. In some embodiments, each image in the training dataset for fine-tuning may be resized (e.g., 299×299 pixels) to be compatible with the original dimensions of the Inception v3 network architecture. Transfer learning can leverage the natural-image features learned by the ImageNet pre-trained network. In some embodiments, the CNN 402 may be trained using back propagation and all layers of the network may be fine-tuned using the same global learning rate of 0.001. As discussed above, the CNN 402 may be configured to provide the probability value of the tested sample as being positive or negative. In FIG. 4, the data flow is from left to right, namely, an image 404 of a microchip with bubbles is subsequently warped into a probability 406 of infection with the target virus using the CNN model 402.

As discussed above, the nanoparticles used for labeling the captured target virus particles on the surface of a microchip may be metal nanoparticles (e.g., PtNPs) and the nanoparticle solution may be a nanoprobe solution that includes nanoparticles modified with a probe material (e.g., antibodies, DNA/RNA probes, aptamer), for example, a Pt-nanoprobe solution that includes PtNPs modified with monoclonal antibody (mAb) against a target virus. FIG. 5 illustrates an example process for preparing a nanooprobe solution in accordance with an embodiment. In the example process 500 illustrated in FIG. 5, the target virus is the Zika virus (ZIKV), the nanoparticles are PtNPs and the probe material is a monoclonal antibody (mAb). As illustrated in FIG. 5, Pt-nanoprobes 506 can be prepared with PtNPs 502 and mAb 504 against the envelope protein of ZIKV. Oxidized Zika IgG monoclonal antibody (mAb) 504 can be coupled to PtNPs 502 through a hydrazide reactive crosslinker of PDPH (3-(2-pyridyldithio)propionyl hydrazide) 508 freshly reduced by 20 mM tris(2-carboxyethyl)phosphine (TCEP) and that possesses a terminal pyridinethiol. The reduced PDPH 508 has a free terminal thiol group that binds to the surface of the PtNPs 502 by a thiol-metal bond, forming hydrazide-modified PtNPs that can react with the carbohydrate residue of the oxidized antibody 504. In some embodiments, the antibody may be oxidized using 10 mM of sodium metaperidate for 1 hour at room temperature. In some embodiments, Pt-nanoprobes may be prepared for other target viruses (e.g., HBV, HCV) using monoclonal antibody against the specific target virus (e.g., anti-Hepatitis B Virus Surface Antigen antibody for HBV and anti-Hepatitis C Virus Core Antigen antibody for HCV).

As discussed above, the surface of a microchip may be modified using a probe material such as, for example, monoclonal antibody (mAb) against a target virus protein to capture any virus particles in the sample on the microchip. FIG. 6 is a diagram illustrating an example microchip surface modification 600 in accordance with an embodiment. In the example process 600 illustrated in FIG. 6, the target virus is the Zika virus (ZIKV). The microchip surface may be functionalized and coupled with anti-ZIKV mAb to allow efficient capture and labeling of ZIKV particles on-chip. Anti-ZIKV mAbs may be conjugated to the surface of the chips using a surface chemistry protocol specifically designed to allow efficient directional conjugation of antibodies using polyethylene glycol (PEG) molecules bi-functionalized with terminal thiol and silane group. In some embodiments, a glass surface of the microchip may initially be silanized with PEG and then oxidized antibodies activated with PDPH can be incubated on the surface of the PEG-modified chip to allow the interaction of pyridyldithiol group of PDPH with the free —SH groups on-chip. The silane polyethylene glycol (PEG) thiol can react with the PDPH crosslinker, allowing a directional binding to the free aldehyde group (CHO) in the carbohydrate residue of the oxidized antibody. PEGylation can increase the conformational stability of proteins and resistance to degradation. Therefore, PEG can be used on the surface of the microchips to help stabilizing the antibody activity, avoiding non-specific interactions, easy washing, and to promote the stability of conjugated biomolecules. PEG can act as a flexible arm that provides maximum accessibility of antibody and a higher chance for avid interaction with virus.

As discussed above, the labeling of captured virus particles with nanoparticles can form virus immunocomplexes on the surface of a microchip, for example, target virus particles labeled with PtNPs can form Pt-virus immunocomplexes. FIG. 7 illustrates an example structure of a target virus labeled with nanoparticles in accordance with an embodiment. In the example structure illustrated in FIG. 7, the target virus is the Zika virus (ZIKV). The example structure forms a three-component sandwich immunocomplex 700 of ZIKV particles 702 and Pt nanoprobes (i.e., PtNPs 704 modified with ZIKV mAb 706).

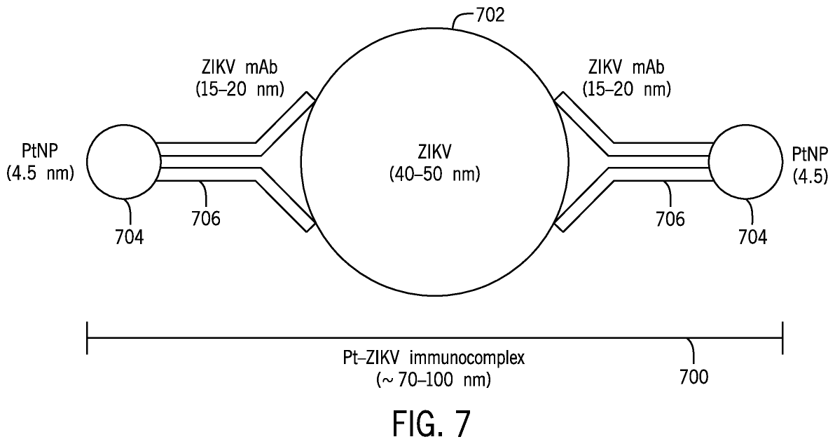

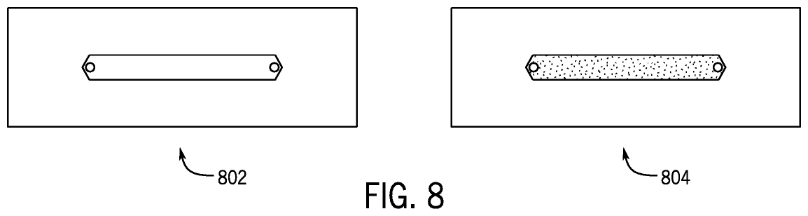

FIG. 8 illustrates an example microchip and sample before bubble formation and after bubble formation in accordance with an embodiment. A first microchip 802 is shown before bubble formation and a second microchip 804 is shown after bubble formation. FIG. 9 shows example images of the formation of bubbles over time in accordance with an embodiment. Example images of bubble formation in a catalyzer solution including H₂O₂ and glycerol at different time points (i.e., from 15-300 s) of incubation, namely, image 902 (15 s), image 904 (30 s), image 906 (60 s), image 908 (120 s), image 910 (240 s), and image 912 (300 s).

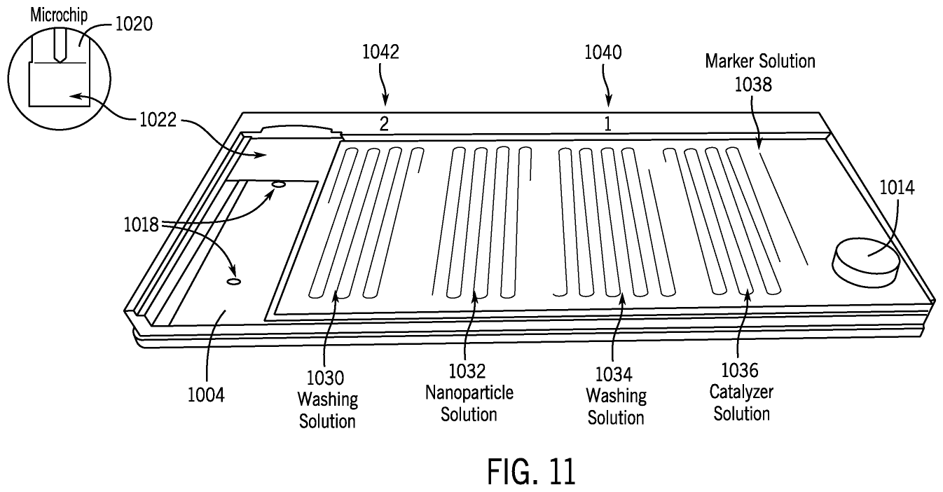

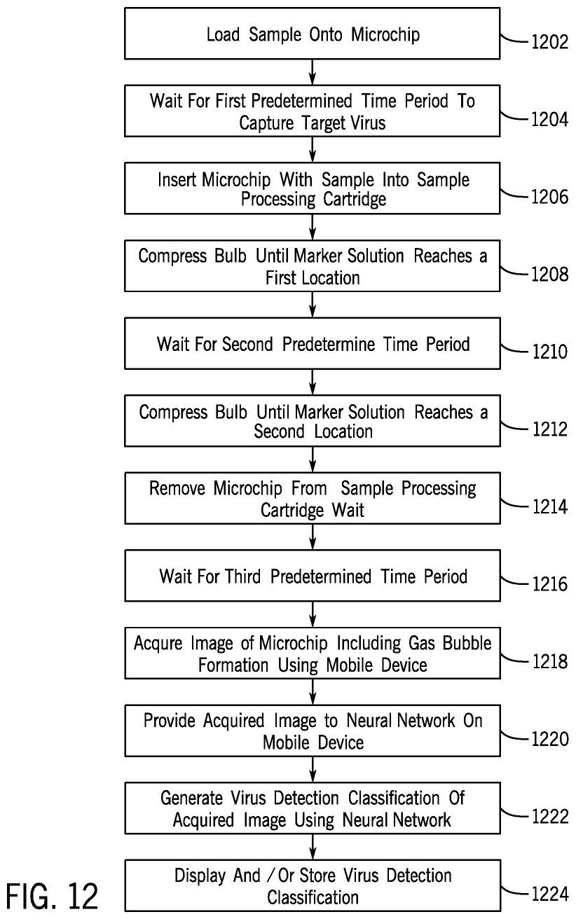

As discussed above, in some embodiments the portions of the bubble assay protocol of blocks 206 to block 216 of FIG. 2 may be performed using a sample processing cartridge that can be preloaded with the reagents and materials needed for sample processing (e.g., including washing solution, nanoprobe solution, catalyzer solution). FIG. 10A is a perspective view of a sample processing cartridge in accordance with an embodiment. In FIG. 10A, a sample processing microfluidic cartridge 1002 includes a microchip insertion slot 1004, a microfluidic core 1006, a top shell layer 1010, a black shell layer 1012, and a control bulb 1014. In some embodiments, the microchip insertion slot 1004 is configured to receive a microchip loaded with a sample from a patient for sample processing. In some embodiments, the microfluidic core consists of poly(methyl methacylate) (PMMA) and double sided adhesive tape (DSA) layers and the top 1010 and back 1012 shell layers may be formed from PMMA. The control bulb may be formed from a flexible material such as, for example, rubber, and may be configured to control the application of various reagents and materials pre-loaded in the cartridge 1002 to a microchip with a sample inserted in the microchip insertion slot 1004 as discussed further below with respect to FIGS. 11 and 12.

FIG. 10B is an exploded view of the sample processing cartridge of FIG. 10A in accordance with an embodiment. In FIG. 10B, the exploded view illustrates a detailed layer structure and the configuration of the main components of the cartridge 1002. In the illustrated embodiment, the microfluidic cartridge 1002 can include four layers of poly(methyl methacylate) (PMMA) assembled together using double-sided adhesive film (DSA), for example three layers of DSA. In particular, the cartridge 1002 includes a microfluidic core 1006 that includes a first core PMMA layer 1006a, a second core PMMA layer 1006b, and a DSA layer 1006c that can include a microfluidic channel 1008. In some embodiments, the microfluidic channel 1008 is a single multi-lane microfluidic channel. In some embodiments, the first 1010 and second 1012 core PMMA layers may be pre-treated with water repellant to be more hydrophobic allowing easy flow through the microfluidic channel 1008. The cartridge 1002 can also include a top PMMA shell layer 1010, a back shell PMMA layer 1012 a control bulb 1014, a cellulose paper pad 1016 and two plastic tips 1018. The top PMMA shell layer 1010 can contains a sample housing cavity which forms the microchip insertion slot 1004. The back PMMA shell layer 10102 may be engraved and modified with the cellulose paper pad 1016.

In some embodiments, the microfluidic channel 1008 may have a total volume of 240 μl. The microfluidic channel 1008 may be terminally connected to a sample in a microchip (not shown) inserted in the microfluidic insertion slot 1004 and the microfluidic channel may be enabled by the control bulb 1014 to enable easy and efficient loading and removing reagents on the microchip. In some embodiments, the cellulose paper pad 1016 may be placed in a waste reservoir to absorb reagents loaded in the microchip channel during sample processing. The two plastic tips 1018 may be located in the first PMMA core layer 1006a and positioned within the microchip insertion slot 1004 when the cartridge is fully assembled. In some embodiments the plastic tips 1008 can be configured to connect the microchip and sample with the reagents when the microchip and sample are inserted into the microchip insertion slot 1004. During assembly of the cartridge 1002, the microfluidic core 1006 may first be loaded with the reagents for sample processing sing a loading well (not shown) on the first PMMA layer 1006a. After the microfluidic core 1006 is loaded with reagents, the top PMMA shell layer 1010 and the back PMMA shell layer 1012 may be added and the control bulb 1014 may be sealed under the top PMMA shell layer 1010 to allow easy and controlled manipulation of the reagents preloaded in the cartridge.

As mentioned, the sample processing cartridge 1002 may be pre-loaded with all of the reagents and materials required for sample processing (i.e., washing solution, nanoparticle solution, and catalyzer solution) which advantageously eliminates manual sample preparation and pipetting of multiple reagents and reducing potential user error. FIG. 11 is a diagram of a sample processing cartridge including reagents and materials for processing a sample of a subject in accordance with an embodiment. A microchip 102 may be inserted into the microchip insertion a lot 1004 of the sample processing cartridge 1002. A marker white zone 1022 may be used for the insertion of the microchip 1020. As mentioned above, two plastic tips 1018 may be positioned within the microchip insertion slot 1004 and can be configured to connect the microchip and sample with the reagents 1030, 1032, 1034, 1036 and 1038 when the microchip and sample are inserted into the microchip insertion slot 1004 In some embodiments, the reagents may be loaded into the cartridge 1002 in the following order: PB solution 1030 (for a first washing step), nanoparticle solution 1032 (e.g., a nanoprobe solution), PB solution 1034 (for a second washing step), catalyzer solution 1036 (e.g., H₂O₂ solution), and a marker solution 1038 (e.g., an indicator dye), separated by air. A first position 1040 on the cartridge 1002 may be identified using a visual identifier on the cartridge housing, for example, a “1” as shown in FIG. 11 and a second position 1042 on the cartridge 1002 may be identified using a visual identifier on the cartridge housing, for example, a “2” as shown in FIG. 11. As described further below with respect to FIG. 12, a control bulb 1014 may be used to control manipulation of the reagents preloaded in the cartridge.

FIG. 12 illustrates a method for virus detection using nanoparticles and a neural network enabled mobile device including processing a microchip with a sample of a subject using the sample processing cartridge of FIG. 11 in accordance with an embodiment. The process illustrated in FIG. 12 is described below as being carried out by the sample processing cartridge 1002 as illustrated in FIG. 11. At block 1202, a sample from a subject may be loaded onto a microchip 1020. In some embodiments, the microchip 1020 may be single channel microchip and the sample can be loaded into the microchip 1020 (e.g., manually using a pipette). As mentioned above, the surface of the microchip can be modified with a probe material such as, for example, monoclonal antibody against a target virus protein (e.g., anti-Zika virus envelope antibody for ZIKV, anti-Hepatitis B Virus Surface Antigen antibody for HBV, and anti-Hepatitis C Virus Core antibody for HCV) to allow efficient capture of particles of the target virus on the surface of the microchip 104. At block 1204, the microchip 1020 with the sample can be incubated for a first predetermined period of time to allow for capture of particles of the target virus if the target virus is present in the sample 106. In one example, the first predetermined time period (or incubation period) can be twenty minutes. At block 1206, the microchip 1020 with the sample may be inserted into the sample processing cartridge 1002, for example, the microchip 1020 may be inserted into a microchip insertion slot 1004 of the cartridge 1002. As mentioned above, the microchip 1020 and sample can be connected to a microfluidic channel 1008 of the microfluidic core 1006 (shown in FIGS. 10A and 10B) using, for example, two plastic tips 1018 in microfluidic core 1006.

At block 1208, a control bulb 1014 of the cartridge 1002 may be compressed to cause the reagents 1030, 1032, 1034, 1036 and 1038 to move through the microfluidic channel 1008 towards the microchip 1020 in the microchip insertion slot 1004 until the marker solution 1038 reaches the first position 1040 on the cartridge 1002 to wash the microchip and sample using the washing solution 1030 (e.g., phosphate buffer) and to load the nanoparticle solution 1032 (e.g., a nanoprobe solution) onto the microchip for labeling of any captured virus particles. At block 1210, the microchip 1020, sample, and nanoparticles (e.g., provided using a nanoprobe solution 1032) can be incubated for a second predetermined period of time to allow any captured particles of the target virus in the sample to be labeled by the nanoparticles. As discussed above with respect to FIG. 2, the labeling of captured virus particles with the nanoparticles can form virus immunocomplexes on the surface of the microchip 1020, for example, target virus particles labeled with PtNPs can form Pt-virus immunocomplexes. In one example, the second predetermined time period (or incubation period) can be twenty minutes.

At block 1212, the control bulb 1014 of the cartridge 1002 may be compressed to cause the reagents 1034, 1036 and 1038 to move through the microfluidic channel 1008 towards the microchip 1020 in the microchip insertion slot 1004 until the marker solution 1038 reaches the second position 1042 on the cartridge 1002 to wash the microchip and sample using the washing solution 1034 (e.g., phosphate buffer) and to load catalyzer solution 1036 (e.g., H₂O₂ solution) onto the microchip 1020 for bubble formation. At block 1214, the microchip and sample may be removed from the sample processing cartridge 1002 and, at block 1216, the microchip 1020, sample, nanoparticle solution 1032 and catalyzer solution 1036 can be incubated for a third predetermined period of time to allow for bubble formation on the surface of the microchip 1020 if there are labeled virus particles on the surface of the microchip 1020. In some embodiments, the third predetermined time period is ten minutes.

At block 1218, after the third predetermined time period, an image of the microchip 1020 and sample, including any gas bubble formations may be acquired using a mobile device, for example, using a camera of the mobile device. At block 1220, the acquired image from block 1218 may be provided to a neural network on the mobile device In some embodiments, the neural network can be trained to generate an output, for example, a virus detection classification, indicating whether the sample is infected (i.e., positive) or not infected (i.e., negative) based on the acquired image of the microchip and sample. The neural network may be trained using known methods. In some embodiments, the neural network is a convolutional neural network (CNN) such as, for example, an Inception v3 architecture, that may be pre-trained using the ImageNet image database. The pre-trained CNN may then be fine-tuned with a training data set that includes pre-labeled images of bubble formations or patterns on microchips using, for example, various target viruses, target virus concentrations, and different dilutions of nanoparticles (e.g., PtNPs). At block 1222, the neural network generates the virus detection classification of the acquired image. For example, in some embodiments, the neural network generates a probability value of the sample being positive or negative for the target virus. At block 1224, the output of the neural network (e.g., the generated probability value(s) and the acquired image of the microchip 104 and sample 106) may be displayed on a display of the mobile device and/or stored in the memory of the mobile device.

Computer-executable instructions for virus detection using nanoparticles and a neural network enabled mobile device according to the above-described methods may be stored on a form of computer readable media. Computer readable media includes volatile and nonvolatile, removable, and non-removable media implemented in any method or technology for storage of information such as computer readable instructions, data structures, program modules or other data. Computer readable media includes, but is not limited to, random access memory (RAM), read-only memory (ROM), electrically erasable programmable ROM (EEPROM), flash memory or other memory technology, compact disk ROM (CD-ROM), digital volatile disks (DVD) or other optical storage, magnetic cassettes, magnetic tape, magnetic disk storage or other magnetic storage devices, or any other medium which can be used to store the desired instructions and which may be accessed by a system (e.g., a computer), including by internet or other computer network form of access

The present invention has been described in terms of one or more preferred embodiments, and it should be appreciated that many equivalents, alternatives, variations, and modifications, aside from those expressly stated, are possible and within the scope of the invention.

1. A system for virus detection in a sample from a subject, the system comprising:
a microchip comprising at least one channel containing the sample from the subject, wherein the sample is processed with nanoparticles and a catalyzer that are configured to generate gas bubbles in the presence of a target virus on a surface of the microchip; and
a mobile device comprising:
a camera configured to acquire an image of the microchip containing the sample from the subject;
a neural network configured to receive the acquired image and to generate a probability regarding the presence of the target virus in the sample from the subject based on the acquired image; and
a display coupled to the neural network and configured to display the probability regarding the presence of the target virus in the sample from the subject.

2. The system according to claim 1, wherein the nanoparticles are metal nanoparticles.

3. The system according to claim 2, wherein the metal nanoparticles are one of platinum (Pt) nanoparticles, gold (Au) nanoparticles, copper (Cu) nanoparticles, iron (Fe) nanoparticles, palladium (Pd) nanoparticles, zinc (Zn) nanoparticles, cadmium (Cd) nanoparticles, and silver (Ag) nanoparticles.

4. The system according to claim 2, wherein the nanoparticles are included in nanoprobes comprising the nanoparticles and a probe material.

5. The system according to claim 1, wherein the nanoparticle are configured to label particles of the target virus.

6. The system according to claim 1, wherein the catalyzer is a catalyzer solution comprising hydrogen peroxide.

7. The system according to claim 1, wherein the mobile device is a smartphone.

8. The system according to claim 1, wherein the neural network is a convolutional neural network.

9. The system according to claim 1, wherein probability regarding the presence of the target virus in the sample is a probability value indicating whether the sample is positive or negative for the target virus.

10. The system according to claim 1, wherein the microchip is modified using a probe material on a the surface of the microchip.

11. A method for virus detection in a sample from a subject, the method comprising:
loading the sample from the subject into a microchip comprising at least one channel;
processing the sample from the subject using at least nanoparticles and a catalyzer that are configured to generate gas bubbles in the presence of a target virus;
acquiring an image of the microchip containing the sample from the subject using a mobile device;
providing the acquired image to a neural network;
generating, using the neural network, a probability regarding the presence of the target virus in the sample from the subject based on the acquired image; and
displaying the probability regarding the presence of the target virus in the sample from the subject on a display.

12. The method according to claim 11, wherein the nanoparticles are metal nanoparticles.

13. The method according to claim 12, wherein the nanoparticles are included in nanoprobes comprising the nanoparticles and a probe material.

14. The method according to claim 11, wherein the nanoparticle are configured to label particles of the target virus.

15. The method according to claim 11, wherein the catalyzer is a catalyzer solution comprising hydrogen peroxide.

16. The method according to claim 11, wherein the mobile device is a smartphone.

17. The method according to claim 11, wherein the neural network is a convolutional neural network.

18. The method according to claim 11, wherein probability regarding the presence of the target virus in the sample is a probability value indicating whether the sample is positive or negative for the target virus.

19. The method according to claim 11, wherein processing the sample from the subject using at least nanoparticles and a catalyzer comprises loading the nanoparticles and the catalyzer onto the microchip using a sample processing cartridge.
